(19) United States
(12) Patent Application Publication
(10) Pub. No.: US 2024/0206442 A1
(43) Pub. Date: Jun. 27, 2024

(54) DEVICE AND ASSEMBLY FOR REARING TERMITES

(71) Applicant: Ynsect, Évry-Courcouronnes Cedex (FR)

(72) Inventors: Thomas LEFEBVRE, Soisy Sur Seine (FR); Qi LI, Combs La Ville (FR)

(73) Assignee: Ynsect, Évry-Courcouronnes Cedex (FR)

(21) Appl. No.: 18/288,483

(22) PCT Filed: Apr. 28, 2022
(86) PCT No.: PCT/FR2022/050817

§ 371 Date: Oct. 26, 2023

(30) Foreign Application Priority Data
Apr. 30, 2021 (FR) FR2104548

(51) Int. Cl. A01K 67/033

(52) U.S. Cl. CPC A01K 67/033

(57) ABSTRACT

The invention relates to a device (1) for rearing termites comprising a tray (2) containing a bed of a substrate (3) suitable for the life of termites, and a set of frames. A lignocellulosic material is placed in each frame (4). The frames are mounted removably side by side in parallel and vertically in the tray such that a lower edge of each frame rests on the substrate bed. The lower edge has one or more openings configured to allow at least some termites to pass between the substrate bed (3) and the interior of the frame (4). The lignocellulosic material of each frame is separated from the lignocellulosic material of the adjacent frame(s) (4) by at least one lateral wall.

The present invention relates to the field of termite rearing. By termites, it should be understood isoptera (Termitoidae/Isoptera).

In particular, the present invention is applicable to all so-called “lower” termites, i.e. having a social determination allowing the production of secondary reproducers or neotenics from larvae or workers or pseudergates. This includes Mastotermitidae, Termopsidae, Hodotermitidae, Kalotermitidae, Rhinotermitidae, Serritermitidae.

The use of termites to convert and valorise lignocellulose-rich residues, in particular from the agro-industry, is an innovative and promising approach. Indeed, termites feature great digestive capacities, including with regard to polymers that are difficult to degrade such as lignin.

Thus, it is possible to profit from the conversion potential offered by termites by rearing termites on an industrial scale.

Devices suitable for experimental rearing and observation of termites are known in the prior art.

For example, the Japanese utility model JP3199484 (U) describes a termite observation frame. This frame includes transparent lateral walls between which sand and wood pieces or chips of various types are placed. An opening formed in the frame enables the introduction of termites and water. This allows observing the behaviour of termites towards the different species of wood placed in the frame. But this laboratory device, suitable for observing the behaviour of termites, is in no way designed or suitable for termite rearing.

Similarly, the document U.S. Pat. No. 2,151,589 describes a container adapted to receive termites and enabling observation thereof. This container consists essentially of a transparent tube in which a blade of a cellulose material is placed in contact with a wall of the tube. This container is suitable for observing a termite colony, but not for large-scale rearing.

In turn, the Chinese utility model CN211153414 (U) describes an insect rearing device. This device is intended for rearing termites in the laboratory, and includes several cells, each cell including a square box and a cavity, and a closure curtain which ensures that no termite escapes. However, this system is suitable only for laboratory rearing, and could be substantially improved.

Finally, the Chinese utility model CN203985616 (U) discloses an incubation device including groups of termite nests (namely Reticulitermes) and allowing visualisation thereof. The device is in the form of a box divided by a plurality of feeding transverse diaphragms into compartments. A cell is formed at the middle of each feeding compartment with a water-retaining material, and a piece of wood is arranged in the cell. However, this device does not properly respect the natural social organisation of termites, and is not suitable for large-scale automated rearing.

Yet, carrying out termite rearing on a large scale is very complicated.

Indeed, the development of intensive termite rearing is a significant challenge primarily due to the social behaviour, constructions and expansion of termites. Unlike the known industrial rearing of dipterans (for example Hermetia illucens) or beetles (for example Tenebrio molitor), isoptera (termites) concentrate in small areas and move in networks of galleries that they construct themselves.

In addition, termites must interact with each other to ensure feeding of castes that are incapable of feeding themselves (soldiers, young larvae, breeders) and more generally to ensure the cohesion of the colony.

Only a few individuals (sometimes a single pair) are responsible for reproduction. For these reasons, a termite farm is barely suitable for regular disturbances, which would be caused during rearing by sorting, harvesting or feeding operations. Hence, termites are organised into complex, socially structured societies, capable of construction, and are therefore difficult to manipulate.

So far, and as the aforementioned documents suggest so, only laboratory-scale termite farms have been used for scientific research.

To date, no known device has proven to be fully satisfactory for carrying out termite rearing on a large scale.

Thus, the invention aims to provide a termite rearing device allowing addressing to all or part of the problems set out hereinabove.

Thus, the invention relates to a termite rearing device including a tray containing a bed of a substrate adapted to the living of termites and a set of frames, a lignocellulosic material being placed in each frame.

The tray forms or contains a support structure of the frames, said support structure holding the frames vertically and removably with respect to the tray. Thus, a lower edge of each frame rests on the substrate bed. The lower edge includes one or more opening (s) configured to enable the passage of at least some termites between the substrate bed and the interior of the frame. The frames are juxtaposed side-by-side substantially parallel, the lignocellulosic material of each frame being separated from the lignocellulosic material of the adjacent frame (s) by at least one lateral wall.

The device thus formed, also called “elementary rearing unit” replicates the natural habitat of termites (in particular termites of the Termopsidae family, in particular Hodotermopsis sjoestedti, or Rhinotermitidae in particular Reticulitermes flavipes). In particular, the arrangement of the frames on a substrate bed replicates the natural environment of these termites. More specifically, the configuration of the frames containing a lignocellulosic material (such as wood, in the form of pieces, chips, compacted powder, sawdust, pulp, or, without limitation, straw, bagasse, cardboard, paper, etc.) but allowing access to and from the substrate on which the frames reproduces a piece of wood with access to the ground (branch or trunk in contact with the ground, or trunk rooted in the ground), which corresponds to the natural habitat of these termites.

Furthermore, the frames thus arranged allow for an easy harvesting of the termites present in said frames, and more generally of the “outputs” of the device (termites, excrement, and nests). The removal of a frame (or of a plurality of frames) also enables easy transfer thereof towards another similar rearing device, in order to create a new termite colony.

Nevertheless, a certain number of termites remain in the substrate, and are not disturbed by harvesting of all or part of the frames and the setting up, where appropriate, of new frames.

Thus, there is always a part of the termite colony which is preserved and which could be used as a reserve to colonise new frames and carry on rearing in the device.

Finally, according to various embodiments of the invention, this configuration enables some farm management operations detailed hereinafter.

Each frame may include two lateral walls.

Each lateral wall may be transparent, for example formed of transparent polycarbonate.

The lateral walls are intended the to hold lignocellulosic material in the frame. Nevertheless, in some embodiments, the material could be held in the frame with no lateral walls to hold it when the frames are removed from the tray. In the case where the walls are transparent, they allow direct observation of the contents of the frame. This observation may be useful during rearing, i.e. during the development of the termite mound in the frame, but also when harvesting the contents of the frame, for example to know if the moment to carry out this harvest has come.

Moreover, the removal of the frames does not lead to the destruction of the galleries present in the frame and in the substrate, the disturbances are limited and enable a characterisation of the evolution of the colony over time, after successive observations of said frames.

Thus, in general, the configuration of the rearing device proposed according to the present invention minimises disturbances to the termite mound and respects its social organisation.

Each lateral wall may have orifices dimensioned so as to enable the passage of at least some termites.

The device may include a system allowing shuttering or clearing all or part of the openings in the lower edge of the frame which rests on the substrate bed.

When the lateral walls are provided with orifices, an adapted calibration of these orifices passing through the walls enables selection of the categories of termites that, according to their development and/or their caste, could penetrate into the considered frame.

Thus, the dimension of the orifices can prevent the circulation of some castes of termites from one frame to another. A size smaller than the head of a soldier or a breeder allows having one (or more) frame (s) that can be colonised only by worker termites. This could prove to be useful when harvesting the frames, or to facilitate “cuttings” by the apparition of secondary (neotenic) reproducers.

Thus, cutting or layering consists in isolating a portion of the termite colony (of some castes) in order to move it into another rearing device, which is empty. To the extent that there are no reproducers (primary or secondary) in the new rearing device, the absence of pheromonal signals emitted by the reproducers allows inducing a differentiation of pseudergates into neotenics (secondary reproducers) and thus starting a new colony.

The system for shuttering the openings in the lower portion of the frame allows access, or not, to the interior of the frame from the substrate. It could complement the orifices present in the lateral walls to enable passage through a frame only for some termites. Indeed, if the edge (i.e. one of the thin edges) of the frame which rests on the substrate has its openings shuttered, the only remaining passages allowing termites to access the interior of the frame are the calibrated orifices in the lateral walls. The shutter system may also be used to selectively close some openings, for example to leave free only openings that enable the passage of only some termites, for example of desired castes. For example, this shutter and clearance system may be in the form of a ruler which may be placed in position or removed from the edge of the frame, or a ruler which can be translated to open or close these openings. For this purpose, the ruler may include corresponding openings, which are placed in correspondence (opposite one another) or not with respect to the openings present in the edge of the frame. Alternatively, this shutter and clearance system may be formed by a removable lower post which can be attached to or removed from the rest of the frame.

Each frame may include on its edge opposite to the lower edge which rests on the substrate bed, a ventilation structure configured so as to enable the passage of air but not the passage of termites.

The ventilation structure may include holes with a maximum diameter of 0.7 mm or slots with a maximum width of 0.7 mm. This structure also allows water supply.

Thus, air ventilation is ensured in the upper portion of the frame, when it is in place in the rearing device. Termite escape, in particular as of the pseudergate stage, can be avoided. Openings that do not let a circle of more than 0.7 mm to be inscribed on their surface, i.e. for example round holes of 0.7 mm at most, or slots with a maximum width of 0.7 mm, allow this.

Furthermore, such openings still let water enter the frame, throughout the ventilation structure.

The lignocellulosic material may contain industrial lignocellulosic residues.

Thus, the residues are converted by digestion by termites. Thus, the considered residues may consist of lignocellulose-rich (or simply cellulose) co-products or waste, derived from wood or plant materials, from the agri-food, agricultural, silvicultural, starch processing, papermaking, etc., industries. As examples, mention may be made of woody residues derived from silviculture, sawdust, bagasse, straw, paper or cardboard waste . . . )

Advantageously, the substrate may contain mainly sand or mineral earth. In particular, the substrate bed may have a thickness comprised between 1 cm and 8 cm.

An objective of the substrate is to replicate a soil in which termites could form galleries, move, and live.

Hence, the substrate bed advantageously consists essentially of a non-nutritive mineral material. Fine sand is suitable for forming this substrate bed. A nutrient material for termites, in particular in the form of powder, such as wood powder, (for example a very fine wood powder, with a grain size in the range of 300 microns) may be integrated into the substrate, in small amounts.

Each frame may have:
a length comprised between 10 cm and 50 cm; for example in the range of 16 cm
a width smaller than the length and comprised between 5 cm and 30 cm; for example in the range of 10 cm;
a thickness comprised between 1 cm and 4 cm, for example in the range of 2 cm.

The tray may be substantially rectangular parallelepiped and have:
a length comprised between 15 cm and 60 cm; for example in the range of 24 cm
a width comprised between 10 cm and 40 cm; for example in the range of 18 cm;
a height comprised between 8 cm and 40 cm, for example in the range of 10 cm.

These dimensions are suitable for the creation of termite colonies. In addition, the frames thus dimensioned are easy to handle, manually or using automated systems. They are suitable for containing easily available wood particles (powder, shavings). In particular, their dimensions could allow keeping the wood particles compacted, and/or another lignocellulosic material, without any particular holding means. The spacing between the lateral walls also allows termites to move. Ideally, this spacing is smaller than twice the diameter of a gallery in order to be able to observe the termites.

The invention also relates to an assembly for rearing termites including several termite rearing devices as described before, said assembly including a vertical structure adapted to receive said rearing devices in the form of shelves or racks. The vertical structure may be organised into parallel shelves, forming aisles therebetween.

The assembly may include an aeraulic system adapted to supply air to each termite rearing device and to extract gases from said assembly.

Thus, the rearing device can be used in large numbers to form a large-scale farm. Advantageously, rearing is “verticalised”, i.e. it uses structures for storing the rearing devices at height. The floor surface used for rearing is reduced, and access to the different rearing devices is simplified. Similarly, the supply of air and water to the rearing devices is simplified. Maintaining adequate rearing conditions (in particular in terms of temperature, humidity, and/or CO₂ level, etc.) could be effectively achieved.

Other particularities and advantages of the invention will appear better in the description hereinafter.

In the appended drawings, given as non-limiting examples:

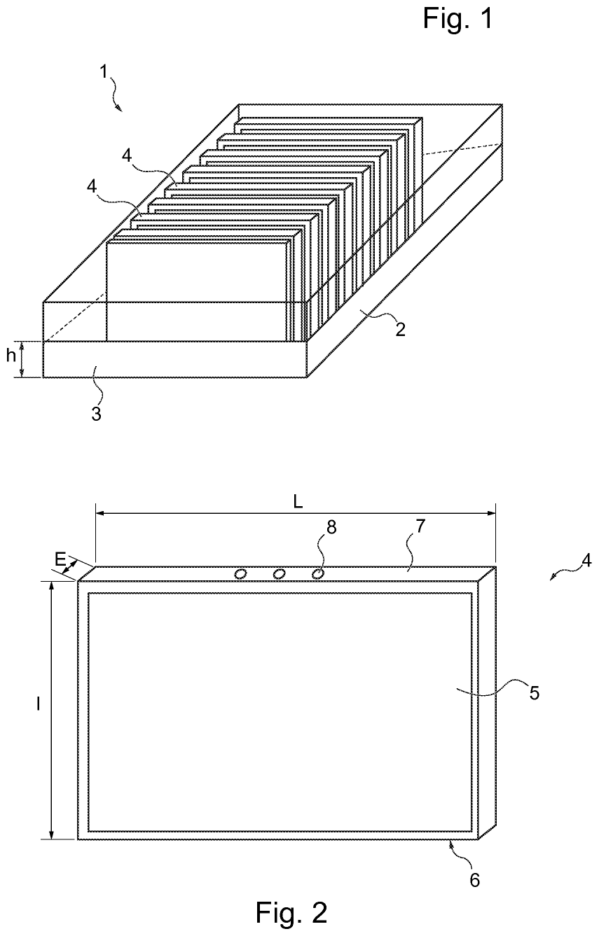

FIG. 1 shows, according to a three-dimensional schematic view, a device in accordance with an embodiment of the invention;

FIG. 2 shows, according to a three-dimensional schematic view, a first example of a frame that can be used in the invention;

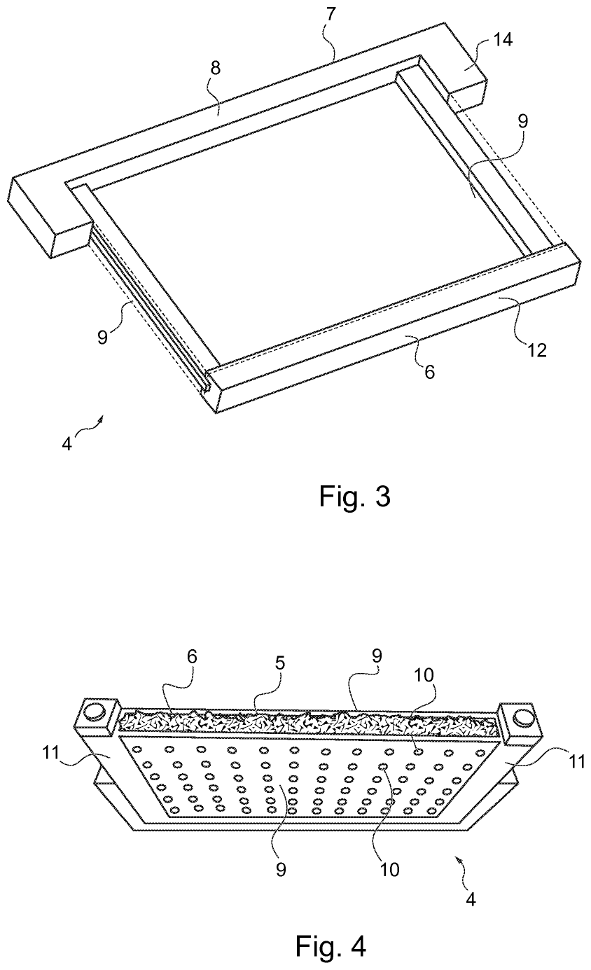

FIG. 3 shows, according to a three-dimensional schematic view, a second example of frame that can be used in the invention;

FIG. 4 shows, according to a three-dimensional schematic view, the frame of FIG. 3;

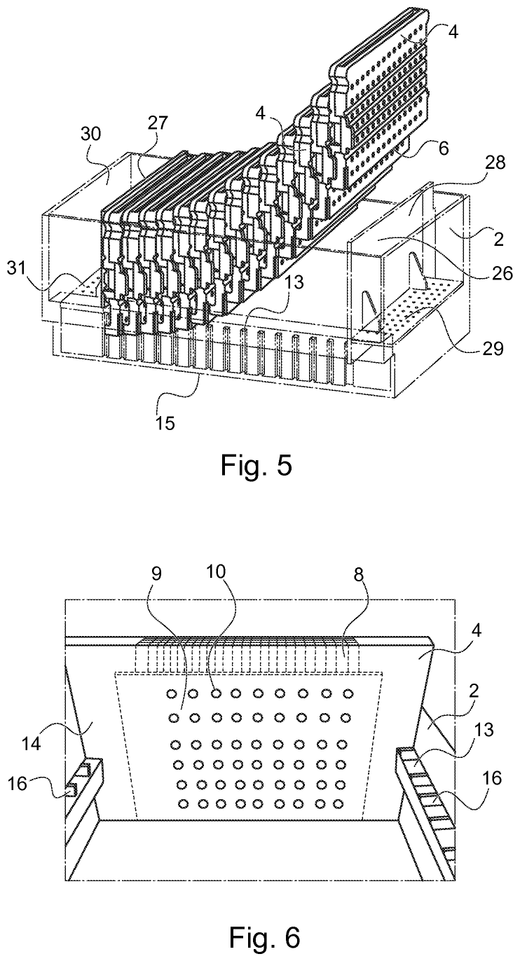

FIG. 5 shows, according to a three-dimensional schematic view, a rearing device in accordance with an embodiment of the invention, including frames in accordance with those of FIGS. 3 and 4;

FIG. 6 shows, according to a three-dimensional schematic view, a frame of FIGS. 3 and 4 installed in a tray in order to form a rearing device in accordance with an embodiment of the invention;

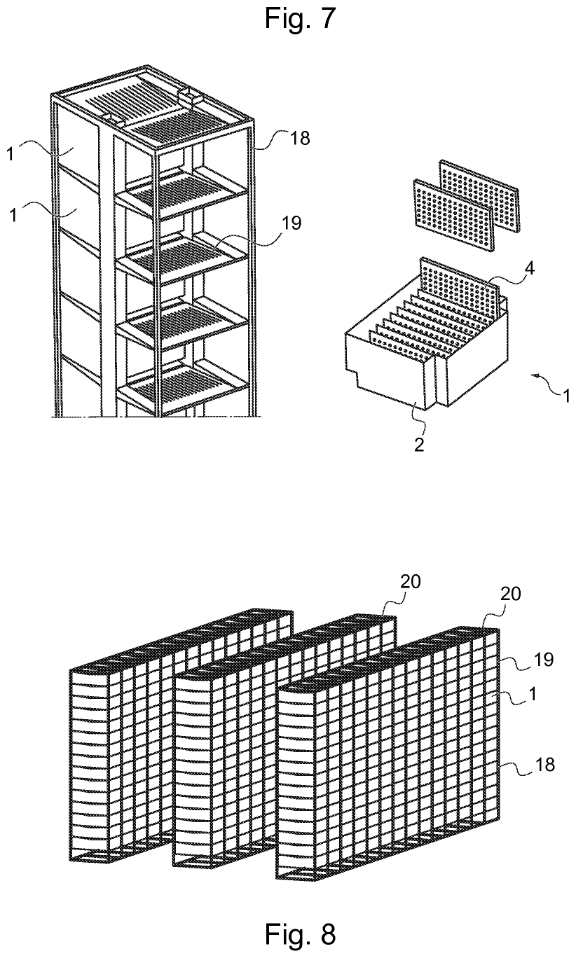

FIG. 7 shows, according to a three-dimensional schematic view, an example of shelving that can be used to form an assembly for rearing termites in accordance with another aspect of the invention;

FIG. 8 shows, according to a three-dimensional schematic view, an example of an assembly for termite rearing in accordance with this aspect of the invention;

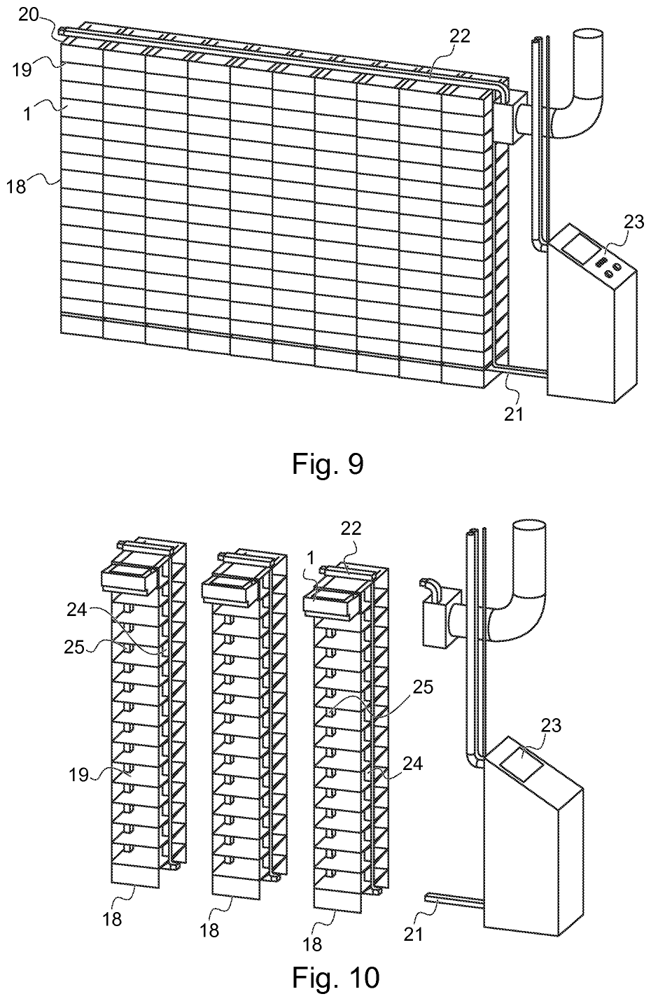

FIG. 9 shows, according to a three-dimensional schematic view, an example of an assembly for termite rearing including a suitable aeraulic system;

FIG. 10 details, according to a three-dimensional schematic view, the assembly of FIG. 9,

FIG. 1 shows, in a three-dimensional schematic view, a termite rearing device 1 in accordance with an embodiment of the invention. This device includes a tray 2. The tray 2 contains a substrate bed 3. The substrate bed consists of an environment, preferably essentially mineral, allowing termites to dig galleries and live in. In particular, the substrate is preferably non-nutritive and non-putrescible, in particular mineral, and advantageously consists of fine particles allowing termites to move and in particular to “manipulate” it with their mandibles and use it to build galleries. This substrate may consist of sand, vermiculite, perlite, etc.

A substrate bed 3 essentially consisting of Fontainebleau sand has been successfully used by the Applicant. Fontainebleau sand (or Fontainebleau micro-sand) is a fine-grain sand (grain diameter smaller than 350 μm), extra-siliceous (97% to 99% silica) and made up of round to sub-angular grains.

For example, the substrate bed may have a bed height h from 1 cm to 8 cm. In particular, a bed height h of 3 cm to 5 cm may be used.

The substrate may be enriched with a material digestible by termites, for example wood powder.

The substrate bed is humidified so as to have a maximum humidity of 25%.

The rearing device 1 further includes a frame assembly 4.

The configuration of the frames 4 is explained in more details with reference to FIG. 2 and FIG. 3 described hereinafter.

Each frame 4 is an element, generally a rectangular parallelepiped, with a small thickness E compared to its width I and its length L.

Each frame contains a lignocellulosic material 5 (for example lignocellulosic) forming a termite-friendly food compound. This compound may include compacted pieces of wood and/or a compacted wood powder. The used wood may be of any species suitable for feeding termites. Common species such as birch, poplar, and pine, and more generally softwood species, may be used. The wood should not have been chemically treated. This food compound may be enriched with industrial lignocellulosic residues, which will thus be treated, converted, by digestion thereof by termites.

The lignocellulosic material is moistened to form a paste which textures the feeding blocks thus formed in the frames 4. Preservatives may be added into the lignocellulosic material to avoid the apparition of mould. For example, potassium sorbate may be added at a concentration of 0.3% without any negative effect on the colony.

The frames 4 are kept vertical, their lower edge 6 resting on the substrate bed 3. By resting, it should be understood that the lower edge 6 is in contact with the substrate bed, and possibly slightly pressed therein. The lower section 6 is provided with one or more opening (s) enabling the passage of at least some termites. By some termites, it should be understood termites having dimensions smaller than a predefined dimension, and which correspond to one or more caste (s) of termites. In the preferred example that is shown, the lower edge 6 is one of the edges located along the length L of the frame.

The frame may include at the upper part, i.e. at the level of its upper edge 7, which is the edge opposite to the lower edge 6, a ventilation structure 8. The ventilation structure enables air exchange between the interior of the frame 4 and the outside, and may have other functions detailed with reference to FIG. 6.

In the rearing device 1, the frames 4 are juxtaposed side-by-side. Thus, they stand substantially parallel in the tray 2 of the rearing device 1.

At least one so-called lateral wall is interposed between the lignocellulosic material contained in a frame and the lignocellulosic material of each adjacent frame.

Thus, at least two embodiments of the frames may be considered. According to a first embodiment, the frames could, in turn, be devoid of lateral walls, like the frame of FIG. 2. This means that the dimensions of the frame and the nature of the lignocellulosic material enable said lignocellulosic material to remain in the frame without any additional holding means. In particular, compacted and humid wood powder features a satisfactory hold, enabling handling of the frames and harvesting thereof without the lignocellulosic material falling from the frame. A lateral wall interposed between the adjacent frames 4 present in the rearing device 1, which therefore forms a spacer between the frames, avoids the passage of termites (or some termites) from one frame to the neighbouring frame.

In the embodiment of FIG. 3, each frame 4 includes two lateral walls 9. In particular, the lateral walls 9 allow ensuring holding of the lignocellulosic material in the frame 4. Advantageously, these lateral walls 9 are transparent, in order to enable observation of the termites and of the part of the colony present in the frame 4.

For example, the lateral walls 9 may be made of transparent plastic, in particular of transparent polycarbonate (polymethyl methacrylate generally so-called plexiglass or plexiglas (registered trademark)).

The lateral walls 9 of the frame 4 may be removably fastened to the frame 4. FIG. 4 shows a frame 4 identical to that of FIG. 3. Frame 4 is shown from below (with its lower edge 6 in the foreground). Thus, FIG. 4 enables a good view of the two lateral walls 9 of the frame 4, as well as of the lignocellulosic material 5 placed between the two lateral walls 9.

The lower edge 6 of the frame includes a large opening (the edge is almost completely open) which allows the circulation of termites from the substrate bed 3 towards the lignocellulosic material of the frame, and vice versa.

The lateral walls 9 include orifices 10 dimensioned so as to enable the passage of at least some termites. In the shown example, the orifices 10 are round orifices which pass through the lateral wall 9. Their diameter is calibrated.

In particular, the diameter of the orifices 10 could prevent the circulation of some castes of termites from one frame to another. For example, a size smaller than that of the head of a soldier or a breeder allows that frames whose lateral walls have such orifices are colonised only by workers.

To avoid the passage of termites through the opening of the lower edge 6 of the frame, the latter is shuttered in this case, for example by means of a suitable ruler (not shown). Alternatively, a removable post 12, such as that shown in FIG. 3, could be used to shutter the lower opening of the frame 4.

Hence, this configuration allows harvesting frames easily. Where appropriate, it enables the creation of a new colony. It also allows renewing food for the colony easily. More generally, this configuration allows sorting the termites concomitantly with harvesting thereof.

Optionally, the lateral walls may be removably mounted on the frame 4. Different removable fastening configurations may be considered. For example, the lateral walls 9 may be accommodated in grooves formed in the lateral posts 11 of the frame 4 and held by stops fastened by screws in said posts.

Where appropriate, the stops may also be used to fasten the shutter ruler of the opening formed in the lower edge 6.

The frames described herein as an example have the following dimensional characteristics: a length L of 16 cm, a width I of 10.4 cm, and a thickness of 2 cm. Of course, other dimensions may be considered, as long as these allow easy handling of the frame, and the development of the termite colony in the frame. In particular, the internal volume of the frames is dimensioned so as to be sufficient to contain a large population of termites and allow the development of secondary reproducer termites.

FIG. 5 shows, in a three-dimensional schematic view, a rearing device in accordance with an embodiment of the invention. In FIG. 5, the tray 2 of the device is transparent, and the substrate bed has been omitted.

In particular, FIG. 5 allows visualising holding of the frames 4 parallel to each other in the tray 2. To this end, the tray 2 includes support structures 13 shaped so as to receive support from the lateral extensions 14 of the frame 4. The support of the lateral extensions 14 of the frame 4 on the support structures 13 keeps the lower edge 6 of the frame 4 at a distance from the bottom 15 of the tray 2.

Support structures 13 are also visible in FIG. 6 which shows the frame of FIGS. 3 and 4 installed in a tray 2 in order to form a rearing device in accordance with an embodiment of the invention. The support structures 13 of the shown example are formed of two longitudinal bars carrying cleats 16 which delimit the position for receiving the frames 4. Of course, many other support structures may be considered. In particular, the support should be adapted to the configuration of the used frames 4, and allow keeping the vertical frames in the tray 2, at a distance from the bottom 15 of the tray 2.

FIG. 6 also illustrates an example of a ventilation structure 8. The ventilation structure 8 is formed in an upper post 17 of the frame 4.

A first function of the ventilation structure is to enable gas exchanges between the interior of the frame (containing the lignocellulosic material) and the exterior of the frame.

It could also enable a water supply to the lignocellulosic material. It may be configured to prevent the passage of termites (particularly as of the pseudergate stage).

For this purpose, the ventilation structure 8 includes, in the example shown herein, ducts with a 0.7 mm side square section (or slightly less). This relevant dimension of 0.7 mm (side for square-shaped holes like in the present example, diameter for circular holes, width for slot-shaped holes) allows achieving these three functions.

FIG. 7 shows, in a three-dimensional schematic view, an example of shelving that could be used to form an assembly for rearing termites in accordance with another aspect of the invention. The assembly includes a vertical structure 18 in the form of a set of shelves 19 adapted to receive rearing devices 1 in accordance with the present invention. As illustrated in FIG. 8, the termite rearing assembly may have many assemblies of shelves (or other vertical structures). These structures may be organised into 20 parallel shelves.

The shelved organisation enables the use of automated systems for placing and removing the rearing devices on the shelves (or any other receiving structure).

An aeraulic system allows supplying air into each of the rearing devices.

An example of an assembly including such an aeraulic system is shown in FIG. 9.

The air supply may be done by establishing an air stream between the shelves 20 and there throughout, and/or thanks to ducts formed directly in the vertical structures 18 for receiving the rearing devices.

In the example shown herein, the ventilation system includes a supply duct 21. The supply conduit 21 introduces air, advantageously having a temperature around 26° C. and advantageously containing around 80% humidity.

The aeraulic system includes a recovery conduit 22.

Control means 23 of the aeraulic system enable control thereof.

As shown in FIG. 10, the rearing assembly, and therefore the aeraulic system, may be modular. In particular, several vertical structures 18 can be joined together to form said assembly. As regards the aeraulic system, the supply duct and the recovery duct 22 may be formed of as many portions of abutting ducts as there are vertical structures 18 in the assembly.

In the shown example, the aeraulic system includes an air supply column 24 and a gas recovery column 25 in each vertical structure 18. The air coming from the supply duct 21 is distributed in each of the air supply columns 24. The air supply column 24 enables the distribution of air at the level of each shelf 19 and in each of the rearing devices 1 of a vertical structure 18. The gas recovery column 25 enables the extraction of air (and where appropriate other gases produced by the termites) from each of the rearing devices of a vertical structure 18. The gases coming from the different air recovery columns are collected in the recovery duct 22.

Air circulation in the termite farm, at the level of each rearing device, is important. It allows maintaining the temperature around a desired setpoint temperature. The temperature is ideally at least 25° C., for example 26° C.

Thus, the rearing device may be optimised for this air circulation. In the exemplary embodiment shown in FIG. 5, the tray 2 includes two inner walls 26, 27. The first inner wall 26 delimits a first volume 28 intended for the entry of air into the tray 2. The first volume 28 is separated from the substrate present in the tray 2 by a first floor 29. The first floor 29 includes holes enabling the passage of air but preventing the passage of termites.

On the other side of tray 2, the second lateral wall 27 delimits a second volume 30 intended to extract air (and other gases) from the device. The second volume 30 is separated from the substrate present in the tray 2 by a second floor 31. The second floor 31 includes holes enabling the passage of gases but preventing the passage of termites.

The relative humidity should also be monitored and controlled, ideally around 80%. This involves a control of the humidity of the air supplied into the farm, but also a water supply system.

Water can be supplied via a system in which a central pipe distributes water to the different shelves. Afterwards, this water flows naturally into the grooves (visible in FIG. 7) provided to this end in said shelves. The grooves are positioned so that the water that falls therefrom fall directly onto the frames below. Water enters each frame through its upper edge 7, throughout the ventilation structure 8 of the frame.

Alternatively or complementarily, water may be supplied using a water spraying system. Thus, a water supply may be ensured by a spraying system. Its operating rhythm may be predetermined (for example, operating for 10 seconds every hour) and/or controlled by a control intended to correct the measured humidity.

An excessively high relative humidity could be detrimental to the colony, as it could cause the apparition of mites.

Finally, in some variants of the invention, the aeraulic system enables the collection of gases from the rearing devices, for recovery thereof. In particular, the methane produced by the termites could thus be recovered.

These two abiotic factors (temperature and humidity) correspond to the living conditions of termite colonies of the Hodotermopsis genus (or others) in their natural environment yet without integrating daily or seasonal cycles. Nevertheless, these cycles could be integrated into some embodiments. However, controlling the brightness (in order to apply a photoperiod) does not seem to be necessary since termites live sheltered from light. Hence, the bioreactor is placed in the dark, except during handling thereof.

The dampwood termite Hodotermopsis sjostedti has proven to be particularly suited to being raised in a device and an assembly as described hereinabove and objects of the invention.

This termite lives in the tropical areas of Asia. It belongs to the so-called “lower” termites of the Termopsidae family, thus containing protozoa in its microbiota and is capable of forming numerous secondary reproducers to increase the size of its colony. In natural conditions, colonies can be quite populated, reticulated in several nests, located in damp and rotten pieces of wood. The nest type is simple without hard construction or specialised chambers. In this termite, the reproducers (mainly secondary, also called neotenics) are mobile and are not limited to one single royal pair but to several males and females. Larvae in stages 4 to 6 (called pseudergates) are capable of developing functional genital members to become neotenic. All these characteristics, which demonstrate the simplicity of housing and the flexibility of the social organisation of this termite, make Hodotermopsis sjostedti a termite suitable for rearing in devices and in an assembly as proposed in the present invention.

Thus, the developed invention allows providing a rearing device whose design allows combining several advantages in the context of termite rearing. In particular, this device allows imitating the natural living conditions of termites by optimising the environmental parameters (temperature, humidity) and by complying with the general structure of a natural nest (including a piece of wood with access to the ground). The proposed allows facilitating the removal of termites from the farm, in their environment, without causing significant disturbance to the rest of the colony. The use of removable frames provides a simple and effective technical solution for harvesting termites and more generally for management of the colonies. In particular, this allows adapting the farm to the expansion of the colony, for example by replacing some frames or by adding new frames.

The configuration suggested in the invention allows modulating the structure of the termite colony by placing spacers (lateral walls) capable of filtering certain castes of termites. In particular, this makes it possible to only give access to certain frames to certain castes of termites. This can be used to create new colonies from these frames where termites are sorted. Finally, certain frames can be removed from a rearing device in order to remove certain elements likely to be affected by diseases or parasites.

In addition to these advantages, a rearing system formed by multiple rearing systems allows for a great modularity and offers advantages in terms of monitoring populations and collecting data relating to rearing. Thus, the rearing configuration suggested in the invention allows sampling the termites while limiting disturbances to the colony. In particular, the use of frames enables access to termites without destroying the galleries. For example, it is possible to quantify the size of a colony through a capture-recapture method, or to identify the presence of reproducers.

It is also possible to estimate the rate of expansion of the colonies or of food consumption, or the efficiency of food conversion done by termites. The configuration also enables a progressive modification (or not) of the termite diet, in particular in order to integrate new types of industrial residues to be treated in the diet of some termite colonies.

1. A termite rearing device (1) including
a tray (2) containing a bed of a substrate (3) adapted to the living of termites;
a set of frames (4), a lignocellulosic material (5) being placed in each frame (4);
the tray (2) forming or including a support structure (13) of the frames (4), said support structure (13) holding the frames vertically and removably with respect to the tray, so that a lower edge (6) of each frame (4) rests on the substrate bed (3),
said lower edge (6) including one or more opening(s) configured to enable the passage of at least some termites between the substrate bed (3) and the interior of the frame (4),
the frames (4) being juxtaposed side-by-side substantially parallel, the lignocellulosic material (5) of each frame being separated from the lignocellulosic material (5) of the adjacent frame(s) (4) by at least one lateral wall (9).

2. The termite rearing device according to claim 1, wherein each frame (4) includes two lateral walls (9).

3. The termite rearing device according to claim 2, wherein each lateral wall (9) is transparent.

4. The termite rearing device according to claim 2, wherein each lateral wall (9) includes orifices (10) dimensioned so as to enable the passage of at least some termites.

5. The termite rearing device according to claim 1, including a system allowing shuttering or clearing all or part of the openings of the lower edge (6) of the frame (4) which rests on the substrate bed (3).

6. The termite rearing device according to claim 1, wherein each frame (4) includes on its edge opposite to the lower edge (6) which rests on the substrate bed (3), a ventilation structure (8) configured so as to enable the passage of air but not the passage of termites.

7. The device according to claim 1, wherein the lignocellulosic material (5) includes industrial lignocellulosic residues.

8. The device according to claim 1, wherein the substrate primarily contains sand or mineral earth.

9. The termite rearing device according to claim 1, wherein each frame (4) has:
a length (L) of 10 cm to 50 cm;
a width (I), smaller than the length (L), of 5 cm to 30 cm;
a thickness (E) of 1 cm 4 cm.

10. The termite rearing device according to claim 1, wherein the tray (2) is substantially rectangular parallelepiped and has:
a length of 15 cm to 60 cm;
a width of 10 cm to 40 cm;
a height of 8 cm to 40 cm.

11. An assembly for rearing termites including several termite rearing devices (1) according to claim 1, said assembly including a vertical structure (18) adapted to receive said rearing devices (1) in the form shelves (19) or racks.

12. The assembly according to claim 11, including an aeraulic system adapted to supply air to each termite rearing device (1) and to extract gases from said assembly.

13. The termite rearing device according to claim 3, wherein each lateral wall (9) is formed of transparent polycarbonate.

14. The termite rearing device according to claim 1, wherein each frame (4) has:
a length (L) of about 16 cm;
a width (I) of about 10 cm;
a thickness (E) of about 2 cm.

15. The termite rearing device according to claim 1, wherein the tray (2) is substantially rectangular parallelepiped and has:
a length of about 24 cm;
a width of about 18 cm;
a height of about 10 cm.
